(19) United States
(12) Patent Application Publication
(10) Pub. No.: US 2004/0044073 A1
(43) Pub. Date: Mar. 4, 2004

(54) Composition and process for treating acne

(75) Inventor: Andrew B. Arata, Lake City, FL (US)

Correspondence Address: Robert F. Frijouf, Frijouf, Rust & Pyle, P.A., 201 East Davis Boulevard, Tampa, FL, 33606, US

(73) Assignee: Innovative Medical Services, El Cajon, CA

(21) Appl. No.: 10/232,499

(22) Filed: Aug. 31, 2002

(51) Int. Cl. C07F 1/10; A61K 31/28

(52) U.S. Cl. 514/495; 556/117

(57) ABSTRACT

A composition and process is disclosed for treating an acne infection with a composition of electrolytically generated silver citrate. The composition of electrolytically generated silver citrate may be applied topically to the acne infection in an aqueous solution or may be incorporated within a cosmetic product., The acne composition may be combined with a composition component such as a cosmetic, a lotion, an emulsion, a gel or a soap.

### BACKGROUND OF THE INVENTION

1. Field of the Invention

This invention relates to acne and more particularly to an improved composition and process for treating Propionibacterium acnes commonly referred to as P. acnes.

2. Description of the Related Art

Acne is a term for a medical condition of plugged pores typically occurring on the face, neck, and upper torso. The plugged pores result in blackheads, whiteheads, pimples or deeper lumps such as cysts or nodules. Severe cases of acne can result in permanent scarring or disfiguring.

Acne is associated with both males and females during adolescence or puberty. Typically, acne begins between the ages of ten and thirteen and usually lasts for five to ten years. For the majority of the population, acne dissipates naturally by the early twenties. For a minority of the population, acne is severe enough to require some treatment by a physician.

Acne occurs when the oil glands of the skin called sebaceous glands produce an increased amount of oil. The sebaceous glands are connected to canals in the skin called hair follicles that terminate in openings in the skin called pores. The increased amount of oil secreted by the sebaceous glands is caused by an increase in androgen hormones in both males and females during adolescence or puberty. Accompanying the increase in the amount of oil secreted by the sebaceous glands is an increase in the shedding of the skin lining the hair follicles. The increase in the amount of secreted oil in combination with the increase in the shedding of the skin lining the hair follicles increases the likelihood of the pores being clogged by the shedding skin. A pore clogged by the shedding skin is referred to as a comedo.

A bacteria naturally occurring on the skin is known as propionibacterium acnes (P. acnes) normally reside on the skin. The propionibacterium acnes invade the clogged follicles and grow in the mixture of oil and cells in the hair follicle. The propionibacterium acnes produce chemicals that stimulate inflammation resulting in acne. Acne lesions range in severity from blackheads, whiteheads and pimples to more serious lesions such as deeper lumps, cysts and nodules.

In many instances, the inflammation within the acne lesion provides an opportunity for secondary infections to invade and grow in the inflamed hair follicle. Some of these secondary infections can be more serious and more resistant to treatment than the primary propionibacterium acnes infection.

Many treatments and medications have been proposed by the prior art to control, reduce or eliminate acne. Among the more conventional topical medications include acne lotion containing benzoyl peroxide, salicylic acid or sulfur. Among the more conventional oral medications include tetracycline, doxycycline, minocycline or erythromycin. In severe cases, isotretinoin is prescribed oral when the severe acne has not responded to other medication. It is believed these antibiotics reduce the propionibacterium acnes in the hair follicle to reduce the underlying cause of the acne.

Among the more unconventional treatments and medications proposed by the prior art to control, reduce or eliminate acne include a light chemical peel. In a light chemical peel, glycolic acid and other chemical agents are applied to the skin to loosen blackheads and decrease acne papules. Other unconventional treatments proposed by the prior art to control, reduce or eliminate acne include the use of ultraviolet light therapy.

Others in the prior art have suggested the use of a citrus component in medications for effecting various beneficial results. Some in the prior art have used citrus components as an ingredient for various products such as cosmetic product and/or other medicinal productions. Among the prior art medications suggesting the use of a citrus component are the following United States Letters Patents.

U.S. Pat. No. 4,021,578 to Harich et al. discloses grapefruit pulp as reacted with an alcohol or ketone, preferably a polyhydric alcohol such as propylene glycol or glycerin, in the presence of a free radical initiator such as ultraviolet light to produce a stable reaction product useful as an ingredient in various cosmetic products and for other purposes.

U.S. Pat. No. 4,264,592 to Xhajanka discloses a citrus fruit fresh cream cosmetic manufactured from the solid parts of citrus fruit. Equal quantities of solid parts of citrus fruit and fresh drinking water are blended and homogenized to produce an emulsion as a colloidal dispersion of all the constituents of the fruit in the fresh water. The citrus fruit fresh cream is a cleanser-emulsion by the action of water reducing its surface tension.

U.S. Pat. No. 4,297,374 to Wess discloses a skin moisturizing and cleansing cream produced by blending a quantity of fresh bananas or avocados with smaller amounts of baking powder, orange juice and solid or liquid vegetable shortening. The ingredients are blended to a creamy texture, and the cream is massaged into the skin by hand. Thereafter, the skin can be wiped with a dry paper or cloth towel, rinsed with warm water or, alternatively, the cream can be allowed to stay on the skin following the massaging. The skin cream is stored in a refrigerator between uses thereof.

U.S. Pat. No. 4,889,844 to Silvetti, Sr., et al. discloses a fructose containing wound healing preparation comprising at least one pharmaceutically acceptable monosaccharide containing from about 3 to 7 carbon atoms and a pharmaceutically acceptable film forming agent.

U.S. Pat. No. 5,063,062 to Greenspan et al. discloses a cleaning composition for cleaning the skin containing orange oil, a pharmaceutically acceptable moisturizer and an emulsifying agent. Preferably the orange oil accounts for between 5% and 60% by volume, and it is further preferred that the composition contains 40% orange oil by volume. The moisturizer is either glycerin, aloe vera, jojoba oil, safflower oil or a combination thereof. The emulsifying agent preferably is oatmeal. The composition is constituted to have a pH of between 4.5 and 6.0, and the composition may be packaged as moistened towellets in hermetic packets.

U.S. Pat. No. 5,177,065 to Silvetti, Sr., et al. discloses a monosaccharide containing wound healing preparation comprising either fructose or ribose, starch hydrolysate and a film forming agent other than starch hydrolysate. The composition is characterized by a weight ratio of fructose or ribose to starch hydrolysate in the range of between about 1:99 and about 15:85, is employed in a method of treating wounds in a host in need of such treatment. In this method a therapeutically effective amount of the composition contacts the wound for a period of time sufficient to initiate wound healing.

U.S. Pat. No. 5,362,714 to Radford et al. discloses a process for transforming and removing latent sediment-forming components from citrus oils to supplement a dewaxing process for the citrus oils. Psoralen epoxides in citrus oils are converted to diols in an accelerated and controlled manner. The epoxide-to-diol transformation is effected by mixing citrus oil with an aqueous acidic treatment solution under conditions (i.e., solution pH and volume) and for a sufficient period of time to convert substantially all of the psoralen epoxides. The diols formed thereby are precipitated and/or preferentially dissolved in the aqueous phase during mixing. Upon separation of the aqueous phase from the oil, the citrus oil is substantially free of psoralen epoxides. By this method, epoxides are inexpensively eliminated from the citrus oils, resulting in an improved product having, for example, reduced sedimentation potential and reduced phototoxicity.

U.S. Pat. No. 5,660,840 to Pruett discloses a system for moisturizing the skin and decreasing established acne. The system consists of a therapeutic preparation comprised of bananas, orange juice, and coconut milk. An alternative procedure consists of the use of a conventional cleanser, toner, facial scrub, and moisturizer. These preparations provide moisturizing benefits, perform a skin sloughing treatment, and alleviate many undesirable skin conditions, including acne. The preparation is used with a facial mask that seals the therapeutic skin treatment on the skin and protects the skin from bacteria and contaminants.

U.S. Pat. No. 5,962,517 to Murad discloses a pharmaceutical composition and method for treating acne having an acne reduction component in an amount sufficient to reduce the redness and blemishes associated with acne. The invention also relates to pharmaceutical compositions having, in addition to the acne reduction component, a skin cell conditioning component in an amount sufficient to properly regulate the keratin and sebum production of the skin cells, thereby inhibiting the appearance of acne. In a preferred form, the skin cell conditioning component is a chromium component. In another preferred form, the composition further includes at least one of a vitamin C source, burdock root, yellow dock root, horsetail extract, a catechin-based composition, a vitamin B.sub.1 source, a vitamin B.sub.2 source, a vitamin B.sub.3 source, a vitamin B.sub.5 source, and a vitamin E source. In a more preferred form, the invention also includes at least one amino acid component, a magnesium component, a selenium component, and biotin. The invention also relates to methods for treating acne by administering, alone or in conjunction with another composition, the pharmaceutical compositions in an amount therapeutically effective in reducing the incidence of acne and methods for additionally inhibiting the appearance of acne by conditioning skin cells.

U.S. Pat. No. 6,017,461 to Garvey et al. discloses a water purification system including a tank fed from a main water supply and an electrolytic ion generator including silver alloy electrodes. A pump circulates water from the tank through the ion generator and back into the tank at a rate of at least 1 L/s to gradually ionize the contents of the tank and generate a concentrate of silver-ion-laden water. An injector feeds the concentrate into the water system as required.

U.S. Pat. No. 6,139,823 to Drescher et al. discloses a citrus fruit fresh cream cosmetic manufactured from the solid parts of citrus fruit. Equal quantity in volume of tenderized solid parts of citrus fruit and fresh drinking water, are blended and homogenized to produce an emulsion as a colloidal dispersion of all the constituents of the fruit in the fresh water. The citrus fruit fresh cream is a cleanser-emulsion by the action of water reducing its surface tension.

In my prior U.S. Pat. No. 6,197,814, I disclosed a novel aqueous disinfectant comprising an aqueous solution of silver citrate wherein the silver is electrolytically generated in a solution of citric acid and water. I hereby incorporate by reference the entire content of my prior U.S. Pat. No. 6,197,814 into the present specification as if fully set forth herein. It is an object of the present invention to utilize the novel aqueous disinfectant to of my prior U.S. Pat. No. 6,197,814 for the treatment of acne.

Therefore, it is an object of the present invention to provide a process for treating an acne infection with a composition of electrolytically generated silver citrate.

Another object of this invention is to provide a process for treating an acne infection with an aqueous solution of electrolytically generated silver citrate wherein silver is electrolytically generated in a solution of citric acid and water.

Another object of this invention is to provide a process for treating an acne infection with a composition of electrolytically generated silver citrate that is effective against secondary infections at the acne infection location.

Another object of this invention is to provide a composition for treating an acne infection with an aqueous solution of electrolytically generated silver citrate.

Another object of this invention is to provide a composition for treating an acne infection with an aqueous solution of electrolytically generated silver citrate incorporated within a cosmetic product.

The foregoing has outlined some of the more pertinent objects of the present invention. These objects should be construed as being merely illustrative of some of the more prominent features and applications of the invention. Many other beneficial results can be obtained by modifying the invention within the scope of the invention. Accordingly other objects in a full understanding of the invention may be had by referring to the summary of the invention, the detailed description describing the preferred embodiment in addition to the scope of the invention defined by the claims taken in conjunction with the accompanying drawings.

### SUMMARY OF THE INVENTION

A specific embodiment of the present invention is described and shown in the attached Detailed Description. For the purpose of summarizing the invention, the invention relates to an acne composition for treating acne comprising an aqueous solution of silver citrate formed by electrolytically generating silver ions within a solution of citric acid and water.

In another example on the invention, the acne composition comprises composition component combined with the aqueous solution of silver citrate to form the acne composition. The composition component may include a cosmetic composition component. In the alternative, the composition component may include a lotion composition component, an emulsion composition component, a gel composition component, a paste composition component, a soap composition component or any other composition component. In a further alternative, the aqueous solution of electrolytically generated silver citrate may be incorporated into currently used products for treating acne.

The invention is also incorporated into the method of process for treating acne comprising forming an acne treatment solution by electrolyticially generating silver citrate by electrolytically generating silver ions within a solution of citric acid and water. The acne treatment solution is topically applied to the site of the acne. In another example of the invention, a composition is formed with the acne treatment solution. The acne treatment solution may be formed into a cosmetic composition.

The foregoing has outlined rather broadly the more pertinent and important features of the present invention in order that the detailed description that follows may be better understood so that the present contribution to the art can be more fully appreciated. Additional features of the invention will be described hereinafter which form the subject of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention.

### BRIEF DESCRIPTION OF THE DRAWINGS

For a fuller understanding of the nature and objects of the invention, reference should be made to the following detailed description taken in connection with the accompanying drawings in which:

FIG. 1 is a diagram of a first method of making an acne treatment composition of the present invention comprising an aqueous solution of electrolytically generated silver citrate;

FIG. 2 is a diagram of a second method of making the acne treatment composition of the present invention;

FIG. 3 is an enlarged detailed view of the ion chamber of FIGS. 1 and 2;

FIG. 4 is an enlarged detailed view of an ion chamber suitable for making the acne treatment composition of the present invention in a batch process;

FIG. 5 is a block diagram of a first further method of processing the aqueous solution of electrolytically generated silver citrate of FIGS. 1 and 2 to be suitable for use as an acne treatment composition;

FIG. 6 is a block diagram of a second further method of processing the aqueous solution of electrolytically generated silver citrate of FIGS. 1 and 2 to be suitable for use as an acne treatment composition;

FIG. 7 is a block diagram of a third further method of processing the aqueous solution of electrolytically generated silver citrate of FIGS. 1 and 2 to be suitable for use as an acne treatment composition;

FIG. 8 is a block diagram of a fourth further method of processing the aqueous solution of electrolytically generated silver citrate of FIGS. 1 and 2 to be suitable for use as an acne treatment composition;

FIG. 9 is an isometric view of a container holding the acne treatment composition in the form of a lotion;

FIG. 10 is an isometric view of a package holding the acne treatment composition in the form of a pre-moistened towel;

FIG. 11 is an isometric view of a package holding the acne treatment composition in the form of a pre-moistened swab;

FIG. 12 is an isometric view of a tube holding the acne treatment composition in the form of a gel;

FIG. 13 is an isometric view of a soap bar holding the acne treatment composition in the form of a solid soap;

FIG. 14 is an isometric view of a jar holding the acne treatment composition in the form of an emulsion; and

FIG. 15 is an isometric view of a ladies compact holding the acne treatment composition in the form of a cosmetic.

Similar reference characters refer to similar parts throughout the several Figures of the drawings.

### DETAILED DISCUSSION

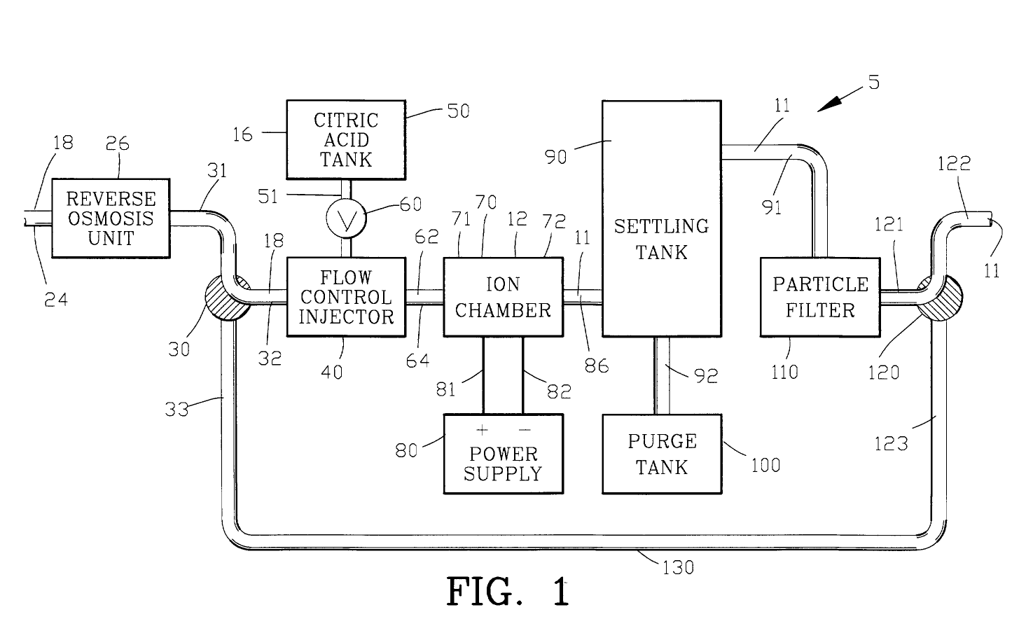

FIG. 1 is a diagram of a first process 5 of making an acne treatment composition 10 comprising an aqueous solution 11 of the present invention for treating acne. The aqueous solution 11 comprises electrolytically generated silver citrate 12 formed by electrolytically generating silver ions 14 within a solution of citric acid 16 and water 18.

The first process 5 is shown as a continuous process of making the acne treatment composition 10. It should be understood that the first process 5 of FIG. 1 is only an example of a process and numerous other variations and/or processes may be utilized to make the acne treatment composition 10 of the present invention.

The first process 5 comprises a water input conduit 24 for introducing water 18 from a water source (not shown) to a water treatment unit shown as a reverse osmosis unit 25. The reverse osmosis unit 26 passes the water 18 from the water input conduit 24 through a semi-permeable membrane (not shown) for removing impurities from the water. Although the water treatment unit is shown as a reverse osmosis unit 26 it should be understood that various water treatment units may be employed within the process shown in FIG. 1. Preferably, the water 18 emanating from the reverse osmosis unit 26 is deionized medically pure water.

The water 18 emanating from the reverse osmosis unit 26 is directed to a valve 30 through a conduit 31. The valve 30 directs the water 18 though a conduit 32 to a flow control injector 40. A citric acid tank 50 contains the concentrated citric acid 16. The concentrated citric acid 16 is directed by a conduit 51 to a metering valve 60 for metering the concentrated citric acid 16 into the flow control injector 40. The flow control injector 40 mixes the concentrated citric acid 16 with the water 18 to provide a dilute citric acid solution 62. The metering valve 60 controls the concentration of the citric acid within the water 18. The dilute citric acid solution 62 is directed into an ion chamber 70 by a conduit 64.

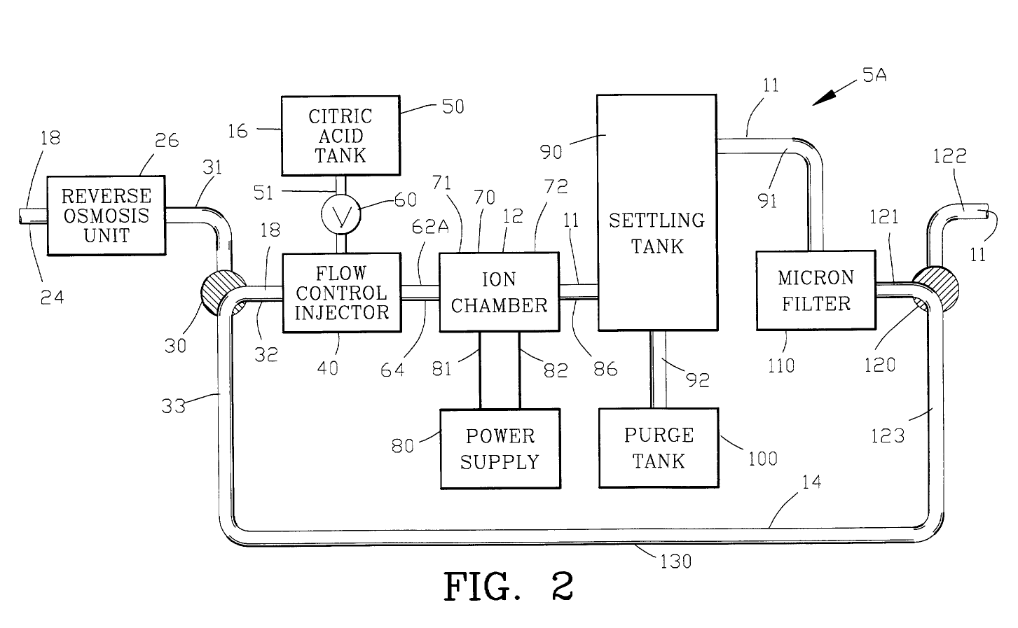

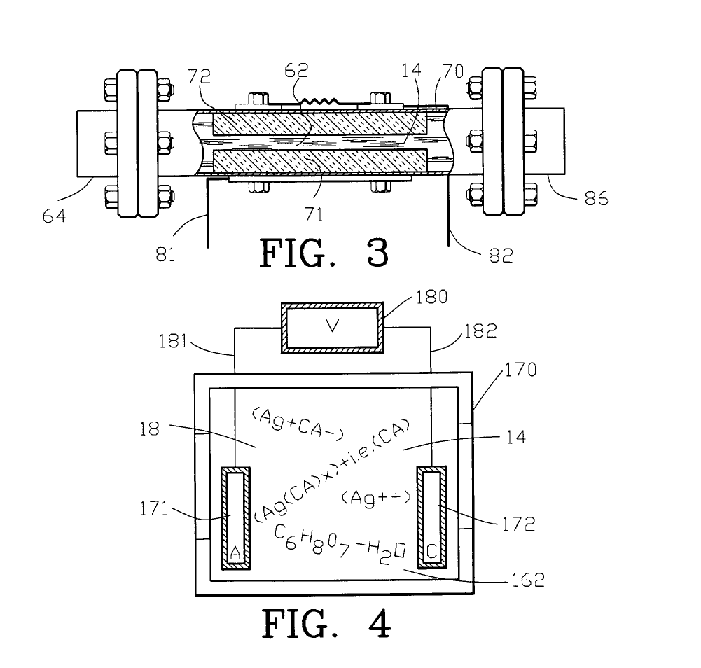

FIG. 3 is an enlarged detailed view of the ion chamber 70 of FIG. 1. The ion chamber 70 includes a positive and a negative electrode 71 and 72. The positive and negative electrodes 71 and 72 are located in a spaced apart position for enabling the diluted citric acid solution 62 to pass between the positive and negative electrodes 71 and 72. Each of the positive and negative electrodes 71 and 72 is fabricated from elemental silver. Preferably, the positive and negative electrodes 71 and 72 are formed from 99.9999% pure elemental silver.

A direct current power supply 80 includes a positive and a negative conductor 81 and 82 connected to the positive and negative electrodes 71 and 72. The positive and negative electrodes 71 and 72 are spaced apart a suitable distance such as 2.0 to 8.0 centimeters to allow an ionic current flow between the positive and negative electrodes 71 and 72.

Upon energizing the direct current power supply 80, an ion current flows between the positive and negative electrodes 71 and 72. The direct ion current flow between the positive and negative electrodes 71 and 72 produces electrolytically free silver ions 14 within the diluted citric acid solution 62. The silver ions 14 react with the citric acid in the diluted citric acid solution 62 to produce the aqueous solution 11 of electrolytically generated silver citrate 12 formed by electrolytically generating silver ions 14 within the solution of citric acid 16 and water 18.

The aqueous solution 11 of electrolytically generated silver citrate 12 is directed by a conduit 86 to a settling tank 90. The settling tank 90 includes an overflow conduit 91 and a drain conduit 92. The acne treatment composition 10 exits the settling tank 90 through the overflow conduit 91. Any precipitated materials from the aqueous solution 11 of electrolytically generated silver citrate 12 within the settling tank 90 fall to the bottom of the settling tank 90. The precipitated materials at the bottom of the settling tank 90 may be removed through the drain conduit 92 to a purge tank 100. The precipitated materials in the purge tank 100 may be recycled.

The aqueous solution 11 of electrolytically generated silver citrate 12 exiting through the overflow conduit 91 from the settling tank 90 is directed to a particle filter 110. Although the particle filter 110 may be any suitable filter, preferably the particle filter 110 is a submicron filter. The filtered aqueous solution 11 of electrolytically generated silver citrate 12 is directed to a valve 120 by a conduit 121. The valve 120 directs the filtered aqueous solution 11 of electrolytically generated silver citrate 12 to a conduit 122 for discharge from the first process 5.

The filtered aqueous solution 11 of electrolytically generated silver citrate 12 may be discharged by the conduit 122 for further processing. In the event a greater concentration of the aqueous solution 11 of electrolytically generated silver citrate 12 is desired, the aqueous solution 11 of electrolytically generated silver citrate 12 may be recirculated for increasing the concentration of the electrolytically generated silver citrate 12 within the aqueous solution 11 of electrolytically generated silver citrate 12.

FIG. 2 is a diagram of a second process 5A of making the aqueous solution 11 of electrolytically generated silver citrate 12 of the present invention in a concentrated form. The second process 5A is shown as a re-circulating process of making the aqueous solution 11 of electrolytically generated silver citrate 12 and for increasing the concentration of the electrolytically generated silver citrate 12. In the concentrated form, the aqueous solution 11 of electrolytically generated silver citrate 12 may be bottled for use at a later time. It should be understood that the second process 5A of FIG. 2 is only an example of a process and numerous other variations and/or processes may be utilized to make the aqueous solution 11 of electrolytically generated silver citrate 12 of the present invention.

In the second process 5A shown in FIG. 2, the valves 30 and 120 are moved into positions opposite to the positions shown in FIG. 1. The valve 120 directs the filtered aqueous solution 11 of electrolytically generated silver citrate 12 to a conduit 123. The conduit 123 is connected through a conduit 130 to the conduit 33 of the valve 30.

The valve 30 directs the filtered aqueous solution 11 of electrolytically generated silver citrate 12 to flow though the conduit 32 into the flow control injector 40. Additional concentrated citric acid 16 is directed through the metering valve 60 into the flow control injector 40. The flow control injector 40 mixes the concentrated citric acid 16 with the filtered aqueous solution 11 of electrolytically generated silver citrate 12 to increase the concentration of the citric acid solution 62A.

The citric acid solution 62A is directed into an ion chamber 70 to produce additional silver ions 14 within the citric acid solution 62A. The silver ions 14 react with the citric acid 16 in the citric acid solution 62A to increase the concentration of the aqueous solution 11 of electrolytically generated silver citrate 12. The aqueous solution 11 of electrolytically generated silver citrate 12 is passed through the settling tank 90 to exit through the overflow conduit 91. The aqueous solution 11 of electrolytically generated silver citrate 12 is filtered by the particle filter 110 and is directed to the valve 120 by the conduit 121.

The valve 30 and 120 are maintained in positions shown in FIG. 2 to continue to recirculate the aqueous solution 11 of electrolytically generated silver citrate 12 for increasing the concentration of the electrolytically generated silver citrate 12. Upon obtaining the desired concentration of the aqueous solution 11 of electrolytically generated silver citrate 12, the valve 120 may be moved to the position shown in FIG. 1 to discharge the aqueous solution 11 of electrolytically generated silver citrate 12 from the conduit 122.

FIG. 4 is an enlarged detailed view of an ion chamber 170 suitable for making the aqueous solution 11 of electrolytically generated silver citrate 12 of the present invention in a batch process. The ion chamber 170 includes a positive and a negative electrode 171 and 172. Each of the positive and negative electrodes 171 and 172 is fabricated from 99.9999% pure elemental silver.

The positive and negative electrodes 171 and 172 are located in a spaced apart position for enabling the citric acid solution 162 to pass between the positive and negative electrodes 171 and 172. Preferably, the positive silver electrode 171 is spaced relative to a negative electrode 172 a distance sufficient to enable the flow of silver ions 14 therebetween. The spacing of the positive and negative electrodes 171 and 172 has been shown in an exaggerated fashion in FIG. 4. Preferably, a spacing of approximately 2.0 to 8.0 mm. has been found to be suitable for the above concentration of citric acid 16 and water 18.

A direct current power supply 180 includes a positive and a negative conductor 181 and 182 connected to the positive and negative electrodes 171 and 172. Upon energizing the direct current power supply 180, a current of silver ions 14 flows between the positive and negative electrodes 171 and 172. The direct current of the silver ions 14 flows between the positive and negative electrodes 171 and 172 produces electrolytically free silver ions 14 within the citric acid solution 162. The silver ions 14 react with the citric acid 16 in the citric acid solution 162 to produce the aqueous solution 11 of electrolytically generated silver citrate 12.

The process of making the acne treatment composition 10 comprises electrolytically generating silver ions 14 in a solution of citric acid 16 and water 18 to form an aqueous solution 11 of electrolytically generated silver citrate 12. Preferably, the aqueous solution 11 of electrolytically generated silver citrate 12 comprises a solution of approximately 5.0% to 10% citric acid 16 in water 18 by weight. A potential difference of 12 volts to 50 volts provides a flow of silver ions 14 in the range of 0.1 amperes to 0.5 amperes per square inch. A more detailed explanation of the content of the solution within the ion chamber 170 will be described in greater detail hereinafter.

The acne treatment composition 10 of the present invention has a stable ionic form having an extended useful shelf-life. The useable shelf-life of the acne treatment composition 10 of the present invention enables the acne treatment composition 10 to be packaged in an aqueous concentrate form.

The improved acne treatment composition 10 comprises the aqueous solution 11 of electrolytically generated silver citrate 12 wherein silver ions 14 are electrolytically generated in a solution of citric acid 16 and water 18. The electrolytically generated silver citrate 12 formed in accordance with the above process has different characteristics than non-electrolytically generated silver citrate.

Concentrations of 0.0001% to 0.003% electrolytically generated silver citrate 12 by weight have been formulated in accordance with the above process. A concentration of 0.003% electrolytically generated silver citrate 12 by weight corresponds to 30 parts per million (ppm).

The Merck Index, Eleventh Edition (1989) page 1348 states that silver citrate is soluble in 3500 parts water. A concentration of 1 to 3500 corresponds to 285 parts per million (ppm). Obviously, the electrolytically generated silver citrate formed in accordance with the above process has different solubility than the non-electrolytically generated silver citrate referred to in the Merck Index.

Nuclear magnetic resonance tests (1H NMR) were preformed on the electrolytically generated silver citrate 12 formed in accordance with the above method 5. The samples showed an overwhelming excess of citric acid 16, with little or no other anions present. It is postulated the silver Ag must be in the form of the cation Ag⁺ complexed with the citric acid 16. It is theorized the empty 5s orbital of Ag⁺ overlaps with the delocalized δ bond on one of the carboxyl groups of citric acid. The citric acid anion is the counterion for this complex ion (Ag(CA)ₓ⁺(Cit)⁻) wherein CA is (C₆H₈O₇—H₂O) and wherein the (Cit)⁻ is (C₆H₇O₇)⁻ and wherein the (C₆H₇O₇)⁻ represents a citrate chemical structure and the (C₆H₈O₇) represents a citric acid structure, and wherein X is an interger.

Another possibility is a Zwitterion, where the negative charge is on the complex itself, (Ag⁺Cit⁻), wherein Cit⁻ is (C₆H₇O₇)⁻ where the total charge of the complex is neutral. Either or both of these species may exist in the silver citrate formed in accordance with the above process. Multiple complexation to Ag⁺ is also possible.

Further speculation based on test data from several types of spectroscopy including UV/Visible absorption and fluorescence spectroscopy, infrared spectroscopy, NMR spectroscopy, and mass spectroscopy indicate that the predominant silver-containing species is a salt containing one silver ion per citrate ion. This salt is not a colloid and but is a weakly bond complex (i.e., an entity containing one or a few silver ions bound to several citrate ions). The chemical formula of the salt may be AgC₆H₇O₇, and may be named silver dihydrogen citrate.

The electrolytically generated silver citrate appears to be different from non-electrolytically generated silver citrate (Ag₃C₆H₅O₇), which is the standard silver citrate salt described in the literature and which contains three silver ions per citrate ion. The solubility of the electrolytically generated silver citrate AgC₆H₇O₇ in water appears to be higher than the non-electrolytically generated silver citrate (Ag₃C₆H₅O₇). The solubility of the electrolytically generated silver citrate AgC₆H₇O₇ in water is 2410 parts per million (PPM) and higher whereas the solubility of the non-electrolytically generated silver citrate (Ag₃C₆H₅O₇) in water is 280 parts per million (PPM).

As contemplated in the present specification, the term electrolytically generated silver citrate includes the silver citrate formed by electrolytically gernerating silver ions in the present of a solution of citric acid and water. Furthermore, the term electrolytically generated silver citrate includes silver dihydrogen citrate.

FIG. 5 is a block diagram of a first further method 200A of processing the aqueous solution 11 of electrolytically generated silver citrate 12 of FIGS. 1 and 2 to be suitable for use as the acne treatment composition 10A. The aqueous solution 11 of electrolytically generated silver citrate 12 is generated in process step 201A. The aqueous solution 11 of electrolytically generated silver citrate 12 may have a concentration between 1 and 10,000 parts per million electrolytically generated silver citrate in water. The aqueous solution 11 of electrolytically generated silver citrate 12 is diluted to form a diluted aqueous solution 11D having a concentration suitable for direct application to the skin surface in process step 202A. The diluted aqueous solution 11D of electrolytically generated silver citrate 12 may have a concentration between 1 to 30 parts per million electrolytically generated silver citrate 12 to water 18. In this example, the diluted aqueous solution 11D forms the acne treatment composition 10A that may be directly applied to the skin surface in process step 203A any suitable manner as should be apparent to those skilled in the art.

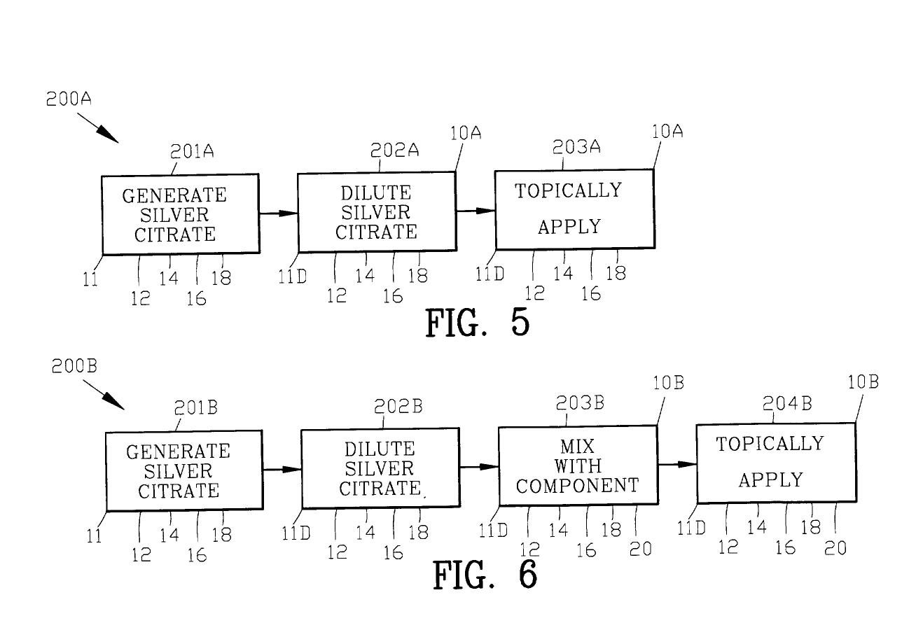

FIG. 6 is a block diagram of a second further method 200B of processing the aqueous solution 11 of electrolytically generated silver citrate 12 of FIGS. 1 and 2 to be suitable for use as an acne treatment composition 10B. The aqueous solution 11 of electrolytically generated silver citrate 12 is generated in process step 201B. The aqueous solution 11 of electrolytically generated silver citrate 12 is diluted to form a diluted aqueous solution 11D having a concentration suitable for direct application to the skin surface in process step 202B. The diluted aqueous solution 11D of electrolytically generated silver citrate 12 is mixed with a component 20 in process step 203B to form the acne treatment composition 10B. The process step 203B of mixing the diluted aqueous solution 11D of electrolytically generated silver citrate 12 with the component 20 may include forming the acne treatment composition 10B in the form of a liquid, a gel, a foam, an emulsion, a paste, a solid or any other suitable form. The acne treatment composition 10B is topically applied to the skin surface any suitable manner.

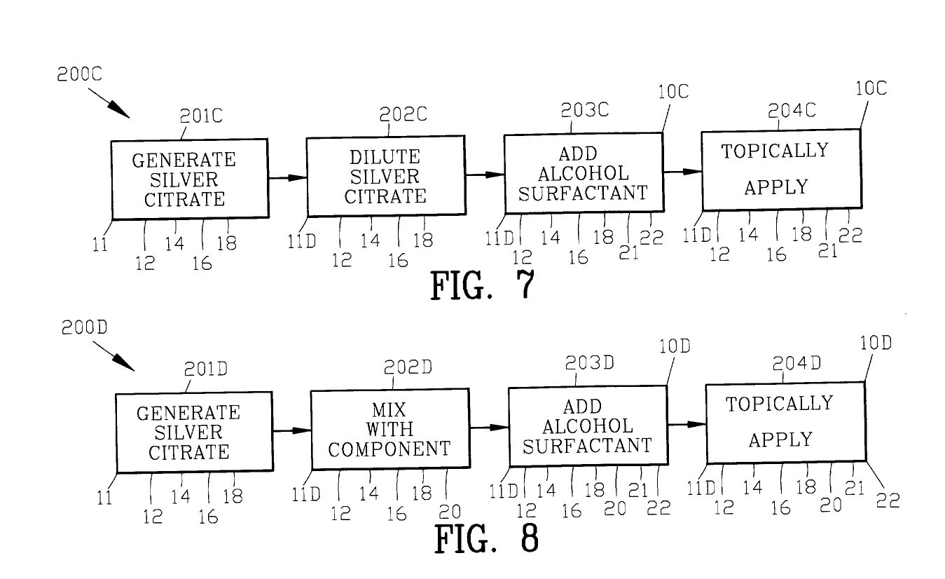

FIG. 7 is a block diagram of a third further method 200C of processing the aqueous solution 11 of electrolytically generated silver citrate 12 of FIGS. 1 and 2 to be suitable for use as an acne treatment composition 10C. The aqueous solution 11 of electrolytically generated silver citrate 12 is generated in process step 201C. The aqueous solution 11 of electrolytically generated silver citrate 12 is diluted to form a diluted aqueous solution 11D having a concentration suitable for direct application to the skin surface in process step 202C. The diluted aqueous solution 11D of electrolytically generated silver citrate 12 is mixed with an alcohol 21 and/or a surfactant or detergent 22 in process step 203C to form the acne treatment composition 10C.

The process step 203C of mixing the diluted aqueous solution 11D of electrolytically generated silver citrate 12 with the alcohol 21 may include mixing the diluted aqueous solution 11D with approximately 1.0 to 20.0% ethyl alcohol by weight. In the alternative, the diluted aqueous solution 11D may be mixed with any suitable alcohol such as ethyl alcohol, isopropal alcohol, or any other suitable alcohol generally used as a modality for treating acne.

The process step 203C of mixing the diluted aqueous solution 11D of electrolytically generated silver citrate 12 with the surfactant or detergent 22 may include mixing the diluted aqueous solution 11D with approximately 0.01% to 2.0% anionic detergent by weight. In the alternative, the diluted aqueous solution 11D may be mixed with any suitable surfactant or detergent 22 such as sodium dodecyl sulfate, triton-X or any orther suitable surfactant and/or detergent. The acne treatment composition 10C is topically applied to the skin surface any suitable manner.

FIG. 8 is a block diagram of a fourth further method 200D of processing the aqueous solution 11 of electrolytically generated silver citrate 12 of FIGS. 1 and 2 to be suitable for use as an acne treatment composition 10D. The aqueous solution 11 of electrolytically generated silver citrate 12 is generated in process step 201D. The aqueous solution 11 of electrolytically generated silver citrate 12 is mixed into a component 20 in process step 202B to dilute the aqueous solution 11 and to form the acne treatment composition 10D.

The process step 202D of diluting the aqueous solution 11 of electrolytically generated silver citrate 12 with the component 20 includes adding only a sufficient amount of the aqueous solution 11 of electrolytically generated silver citrate 12 with the component 20 to have a concentration suitable for direct application to the skin surface in process step 202D. The process step 202D of mixing the aqueous solution 11 of electrolytically generated silver citrate 12 with the component 20 may include forming the acne treatment composition 10D in the form of a liquid, a gel, a foam, an emulsion, a paste, a solid or any other suitable form.

The diluted aqueous solution 11D of electrolytically generated silver citrate 12 is mixed with an alcohol 21 and/or a surfactant or detergent 22 in process step 203D to form the acne treatment composition 10D as set forth above. The acne treatment composition 10D is topically applied to the skin surface any suitable manner.

The invention is also incorporated into a process for treating acne comprising the steps of forming an acne treatment solution 10 by electrolytically generating silver citrate 12 by electrolytically generating silver ions 14 within the solution of citric acid 16 and water 18. The acne treatment solution 10 is formed into the acne treatment solution 10A-10D as shown in FIGS. 5-8. The acne treatment solution 10 is topically applied to the site of the acne.

Table 1 illustrates the efficacy of the acne treatment solution 10 of the present invention against the Propionibacterium acnes commonly referred to as P. acnes. The tests were conducted using a 30-ppm electrolytically generated silver citrate 12 with 5 percent (5%) by weight citric acid 16 and 0.03% by weight SLC in purified water 18. The tests were performed by nationally recognized independent laboratories under Association of Analytical Chemists (AOAC) protocol and Good Laboratory Practices in accordance with EPA regulations.

TABLE I
KILL TIME STUDY FOR PROPIONIBACTERIUM ACNES

| | | | | PERCENT | LOG₁₀ |
|---|---|---|---|---|---|
| SAM- | INITIAL | CONTACT | FINAL | REDUC- | REDUC- |
| PLE | COUNTS | TIME (Sec.) | COUNTS | TION | TION |
| 1 | 1.9 × 108 | 15 | 3 | >99.9998 | 5.80 |
| 1 | 1.9 × 106 | 30 | <1 | >99.99995 | 6.28 |
| 1 | 1.9 × 108 | 45 | <1 | >99.99995 | 6.28 |
| 1 | 1.9 × 108 | 60 | <1 | >99.99995 | 6.28 |
| 1 | 1.9 × 108 | 75 | <1 | >99.99995 | 6.28 |
| 1 | 1.9 × 106 | 90 | <1 | >99.99995 | 6.28 |
| 2 | 1.9 × 106 | 15 | <1 | >99.99995 | 6.28 |
| 2 | 1.9 × 108 | 30 | <1 | >99.99995 | 6.28 |
| 2 | 1.9 × 108 | 45 | <1 | >99.99995 | 6.28 |
| 2 | 1.9 × 108 | 60 | <1 | >99.99995 | 6.28 |
| 2 | 1.9 × 108 | 75 | <1 | >99.99995 | 6.28 |
| 2 | 1.9 × 108 | 90 | <1 | >99.99995 | 6.28 |

30 ppm prepared with 5°/a (w/w) Citric Acid in Purified Water
Intial Count and Final Count in (CFU/ml)

The percent reduction and log reduction results for each sample 1 and 2 of the acne treatment solution 10 can be found in Table 1. The organism titer was determined to be 2.0×10 CFU/mL. The positive control titer was determined to be 1.9×10⁶ CFU/mL. The percent neutralization recovery was 72% for sample 1 and 94% for sample 2.

Table 2 illustrates the efficacy of the acne treatment solution 10 of the present invention against secondary infections caused by staphylococcus aureus commonly referred to as S. aureus. The tests were conducted using a 30-ppm electrolytically generated silver citrate 12 with 5 percent (5%) by weight citric acid 16 and 0.03% SLS by weight in purified water 18. The tests were performed by nationally recognized independent laboratories under Association of Analytical Chemists (AOAC) protocol and Good Laboratory Practices in accordance with EPA regulations.

TABLE II
DISINFECTANT EFFICACY RESULTS STAPHYLOCOCCUS AUREUS

| | | | CARRIERS | CARRIERS |
|---|---|---|---|---|
| | TIME | CARRIERS | EXHIBITING | EXHIBITING |
| SAMPLE | POINT | TESTED | GROWTH | NO GROWTH |
| 1 | 30 seconds | 10 | 9 | .1 |
| | 1 minute | 10 | 7 | 3 |
| | 2 minutes | 10 | 0 | 10 |
| 2 | 30 seconds | 10 | 9 | 1 |
| | 1 minute | 10 | 7 | 3 |
| | 2 minutes | 10 | 0 | 10 |
| Positive | N/A | 2 | 2 | 0 |
| Control | | | | |
| Media | N/A | 3 | 0 | 3 |
| Control | | | | |

FIG. 9 is an isometric view of a container 300A holding an acne treatment composition 310A in the form of a liquid composition component 320A. The container 300A has an opening 302A for discharging the liquid composition component 320A. The liquid composition component 320A may be a viscous liquid such as lotion or the like. The liquid composition component 320A may be cosmetic facial lotion or the like.

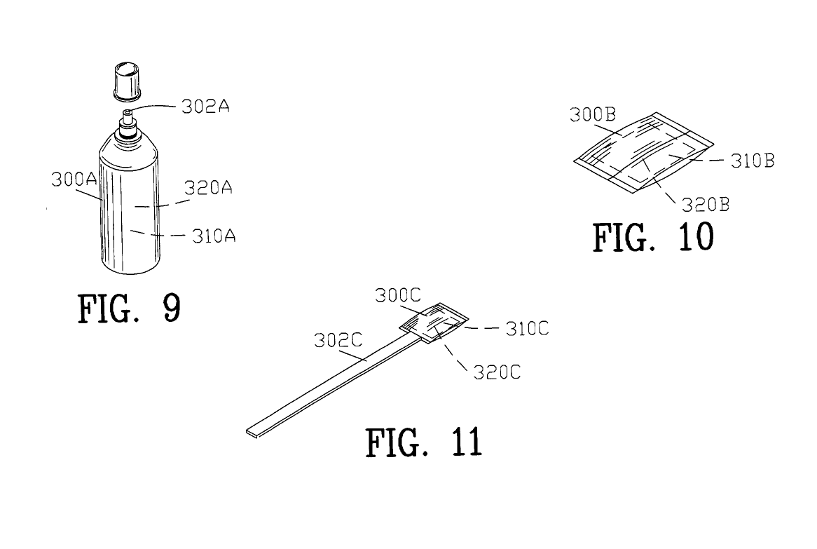

FIG. 10 is an isometric view of a package 300B holding an acne treatment composition 310B in the form of a liquid permeated towel component 320B. The towel component 320B is at least partially saturated with the liquid acne treatment solution 310B. Preferably, the towel component 320B is disposable. The package 300B is a liquid impermeable frangible package 300B for holding the liquid permeated towel component 320B

FIG. 11 is an isometric view of a swab applicator package 300C for an acne treatment composition 310C in the form of a liquid permeated swab component 320C. The liquid permeated swab component 320C is supported by an applicator stick 302C. The swab component 320C is at least partially saturated with the liquid acne treatment solution 310C. Preferably, the swab component 320C is disposable. The swab applicator package 300C is a liquid impermeable frangible package 300C for holding the liquid permeated towel component 320C.

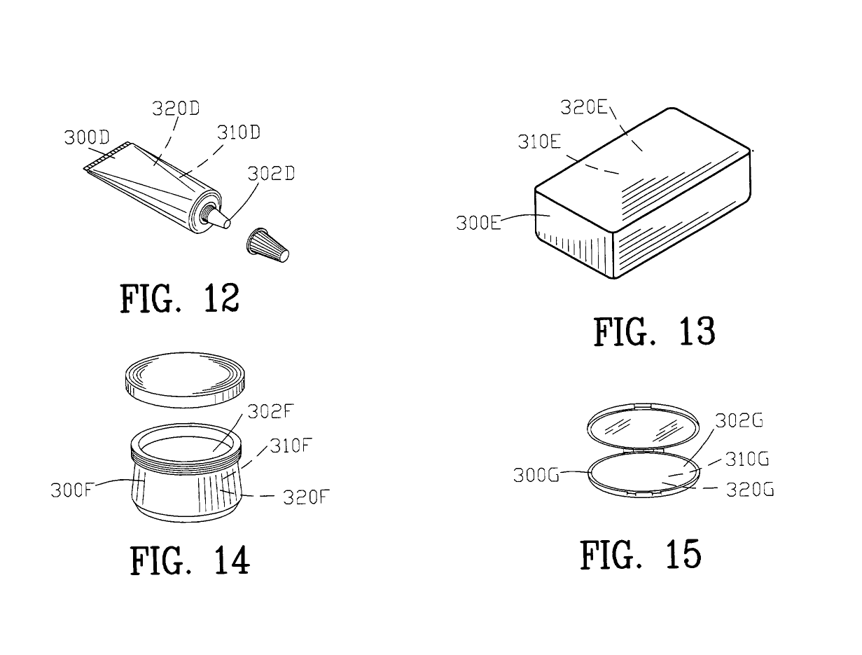

FIG. 12 is an isometric view of a flexible container tube 300D holding an acne treatment composition 310D in the form of a gel composition component 320D. The flexible container tube container 300D has an opening 302D for discharging the gel composition component 320A. The gel composition component 320D may be cosmetic facial lotion or the like.

FIG. 13 is an isometric view of a solid bar of soap 300E containing an acne treatment composition 310E. The acne treatment composition 310E is blended into the solid bar of soap 300E.

FIG. 14 is an isometric view of a container jar 300F holding an acne treatment composition 310F in the form of an emulsion composition component 320F. The emulsion composition component 320F may be a viscous emulsion such as cream or the like. The emulsion composition component 320F may be cosmetic facial cream or the like.

FIG. 15 is an isometric view of a ladies compact 300G holding an acne treatment composition 310G in the form of a paste composition component 320G. The paste composition component 320G may be a facial foundation, facial powder or the like.

The present invention provides an acne treatment composition and process for treating acne. The acne treatment composition comprises electrolytically generated silver citrate in water. A mild surfactant and a suitable alcohol may be incorporated within the acne treatment composition.

The acne treatment composition substantially eliminates the P. acne bacteria within 30 seconds of contact time and provides residual protection against infection. The acne treatment composition safely and effectively removes body oils and dirt away from the affected skin area. Furthermore, the acne treatment composition substantially reduces secondary infection caused by S. aureus and other bacteria. The acne treatment composition helps to eliminate blemish redness, soreness and promotes more rapid healing of epidermal skin cell tissue.

The acne treatment composition may also be used alone or in conjunction with other pharmaceutical compositions synergistically, in an amount that would be a therapeutically superior method of treating acne and eliminating secondary infections caused by S. aureus and other bacteria, promote more rapid reduction in the appearance of acne blemishes, chronic endemic blemish re-occurrence and provides residual protection against re-infection.

The present disclosure includes that contained in the appended claims as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

What is claimed is:

1. A process for treating acne, comprising
forming an acne treatment solution by electrolytically generating silver citrate by electrolytically generating silver ions within a solution of citric acid and water; and
topically applying the acne treatment solution to the site of the acne.

2. A process for treating acne as set forth in claim 1, wherein the step of forming the solution of electrolytically generated silver citrate forms a composition in the form of
(Ag(CA)ₓ⁺(Cit)⁻)
wherein CA is (C₆H₈O₇—H₂O) and wherein the (Cit)⁻ is C₆H₇O₇)⁻ and wherein the (C₆H₇O₇)⁻ represents a citrate chemical structure and the (C₆H₈O₇) represents a citric acid structure, and wherein X is an integer.

3. A process for treating acne as set forth in claim 1, wherein the step of forming the solution of electrolytically generated silver citrate forms a composition in the form (Ag⁺Cit⁻), wherein Cit⁻ is (C₆H₇O₇)⁻.

4. A process for treating acne as set forth in claim 1, wherein the step of topically applying the acne treatment solution includes topically applying the acne treatment solution having a concentration between 1 to 30 parts per million electrolytically generated silver citrate in water to the site of the acne.

5. A process for treating acne, comprising
forming an acne treatment solution by electrolytically generating silver citrate by electrolytically generating silver ions within a solution of citric acid and water;
forming a composition with the acne treatment solution; and
topically applying the composition to the site of the acne.

6. A process for treating acne as set forth in claim 5, wherein the step of forming the solution of electrolytically generated silver citrate includes forming the solution of electrolytically generated silver citrate to have a concentration between 1 and 10,000 parts per million electrolytically generated silver citrate in water.

7. A process for treating acne as set forth in claim 5, wherein the step of forming the composition with the acne treatment solution includes forming a composition with the electrolytically generating silver citrate having a concentration between 1 to 30 parts per million electrolytically generated silver citrate within the composition.

8. A process for treating acne as set forth in claim 5, wherein the step of forming the acne solution includes forming the solution of electrolytically generated silver citrate to have a concentration between 1 and 10,000 parts per million electrolytically generated silver citrate in water; and
the step of forming the composition with the acne treatment solution includes forming the composition with the electrolytically generated silver citrate having a concentration between 1 to 30 parts per million electrolytically generated silver citrate within the composition.

9. A process for treating acne as set forth in claim 5, wherein the step of forming the composition with the acne treatment solution includes forming a cosmetic composition with the acne treatment solution.

10. A process for treating acne as set forth in claim 5, wherein the step of forming the composition with the acne treatment solution includes forming a lotion composition with the acne treatment solution.

11. A process for treating acne as set forth in claim 5, wherein the step of forming the composition with the acne treatment solution includes forming an emulsion with the acne treatment solution.

12. A process for treating acne as set forth in claim 5, wherein the step of forming the composition with the acne treatment solution includes forming a gel composition with the acne treatment solution.

13. A process for treating acne as set forth in claim 5, wherein the step of forming the composition with the acne treatment solution includes forming a paste composition with the acne treatment solution.

14. A process for treating acne as set forth in claim 5, wherein the step of forming the composition with the acne treatment solution includes forming a wipe pre-moistened with the acne treatment solution.

15. A process for treating acne, comprising
forming an acne treatment solution by electrolytically generating silver citrate by electrolytically generating silver ions within a solution of citric acid and water;
forming a cosmetic composition with the acne treatment solution with the electrolytically generated silver citrate having a concentration between 1 to 30 parts per million within the cosmetic composition; and
topically applying the cosmetic composition to a site of the acne.

16. An acne treatment composition for treating acne, comprising
an aqueous solution of silver citrate formed by electrolytically generating silver ions within a solution of citric acid and water.

17. An treatment acne composition for treating acne as set forth in claim 16, wherein the solution of electrolytically generated silver citrate forms a composition in the form of
(Ag(CA)ₓ⁺(Cit)⁻)
wherein CA is (C₆H₈O₇—H₂O) and wherein the (Cit)⁻ is C₆H₇O₇)⁻ and wherein the (C₆H₇O₇)⁻ represents a citrate chemical structure and the (C₆H₈O₇) represents a citric acid structure, and wherein X is an interger.

18. An acne treatment composition for treating acne as set forth in claim 16, wherein the solution of electrolytically generated silver citrate forms a composition in the form (Ag⁺Cit⁻), wherein Cit⁻ is (C₆H₇O₇)⁻.

19. An acne treatment composition for treating acne as set forth in claim 16, wherein the acne treatment solution has a concentration between 1 to 30 parts per million electrolytically generated silver citrate in water to the site of the acne.

20. An acne treatment composition for treating acne as set forth in claim 16, including an absorbent hand towel; and
said absorbent hand towel being at least partially saturated with said acne treatment solution.

21. An acne treatment composition for treating acne, comprising
an aqueous solution of silver citrate formed by electrolytically generating silver ions within a solution of citric acid and water; and
a composition component combined with said aqueous solution of silver citrate to form the acne treatment composition having a concentration between 1 to 30 parts per million electrolytically generated silver citrate in the acne treatment composition.

22. An acne treatment composition for treating acne as set forth in claim 21, wherein said solution of electrolytically generated silver citrate has a concentration between 1 and 10,000 parts per million electrolytically generated silver citrate in water.

23. An acne treatment composition for treating acne as set forth in claim 21, wherein said acne treatment composition has a concentration between 1 to 30 parts per million electrolytically generated silver citrate within the acne treatment composition.

24. An acne treatment composition for treating acne as set forth in claim 21, wherein said solution of electrolytically generated silver citrate has a concentration between 1 and 10,000 parts per million electrolytically generating silver citrate in water; and
said acne treatment composition having a concentration between 1 to 30 parts per million electrolytically generated silver citrate within the acne treatment composition.

25. An acne treatment composition for treating acne as set forth in claim 21, wherein said composition component includes a cosmetic composition component.

26. An acne treatment composition for treating acne as set forth in claim 21, wherein said composition component includes a lotion composition component.

27. An acne treatment composition for treating acne as set forth in claim 21, wherein said composition component includes an emulsion composition component.

28. An acne treatment composition for treating acne as set forth in claim 21, wherein said composition component includes a gel composition component.

29. An acne treatment composition for treating acne as set forth in claim 21, wherein said composition component includes a paste composition component.

30. An acne treatment composition for treating acne as set forth in claim 21, wherein said composition component includes a soap composition component.

31. An acne treatment composition for treating acne, comprising
an aqueous solution of silver citrate formed by electrolytically generating silver ions within a solution of citric acid and water; and
a cosmetic component combined with said aqueous solution of silver citrate to form the acne treatment composition having a concentration between 1 to 30 parts per million electrolytically generated silver citrate in the acne composition.

32. An acne composition for treating acne, comprising:
an aqueous solution of silver citrate formed by electrolytically generating silver ions within a solution of citric acid and water; and
approximately 20% alcohol by weight.

33. An acne composition for treating acne as set forth in claim 32, wherein the alcohol is approximately 20% ethyl alcohol (ETOH) by weight.

34. An aqueous disinfectant as set forth in claim 34, including a suitable detergent/surfactant.

35. An aqueous disinfectant, comprising:
an aqueous solution of silver citrate formed from electrolytically generated silver in a solution of citric acid and water;
approximately 1.0 to 20.0% ethyl alcohol by weight; and
approximately 0.01% to 2.0% anionic detergent by weight.

36. An aqueous disinfectant as set forth in claim 35, wherein the detergent is sodium dodecyl sulfate.

37. An aqueous disinfectant as set forth in claim 35, wherein the detergent is Trion-X 100.
